(19) United States
(12) Patent Application Publication
(10) Pub. No.: US 2026/0108702 A1
(43) Pub. Date: Apr. 23, 2026

(54) ROTARY IMPELLER TYPE AROMATHERAPY HUMIDIFIER

(71) Applicant: Shenzhen Baowu Technology Co., LTD, Shenzhen (CN)

(72) Inventor: Changchun LI, Ganzhou (CN)

(73) Assignee: Shenzhen Baowu Technology Co., LTD, Shenzhen (CN)

(21) Appl. No.: 19/428,274

(22) Filed: Dec. 21, 2025

Related U.S. Application Data
(63) Continuation of application No. PCT/CN2024/087921, filed on Apr. 16, 2024.

(30) Foreign Application Priority Data
Jun. 20, 2023 (CN) 202321582837.2

(51) Int. Cl. A61M 21/02; A61M 11/00; A61M 21/00

(52) U.S. Cl. CPC A61M 21/02; A61M 11/006; A61M 2021/0016; A61M 2205/103; A61M 2205/587

(57) ABSTRACT

The present disclosure provides a rotary impeller type aromatherapy humidifier, including a humidifier main body, a control circuit board and an atomization assembly which are arranged inside the humidifier main body, a mist guide cylinder for transporting the mist generated by the atomization assembly to the mist storage chamber and a rotary impeller arranged close to the mist storage chamber and axially rotates, wherein the upper part of the humidifier main body is provided with a mist storage chamber communicated with the humidifier main body and used for accumulating mist and condensed water droplets thereof, the rotary impeller includes several water-storable water storage lobes, and a water outlet is arranged on the mist storage chamber or the mist storage chamber is connected with a water outlet pipeline.

### CROSS-REFERENCE TO RELATED APPLICATIONS

The application claims priority to Chinese patent application No. 202321582837.2, filed on Jun. 20, 2023, the entire contents of which are incorporated herein by reference.

### TECHNICAL FIELD

The present disclosure relates to the technical field of humidifier products, particularly to a rotary impeller type aromatherapy humidifier which is rational in structure design and high in operation and ornamental value.

### BACKGROUND

Aromatherapy diffusers and humidifiers are common products in people's daily lives, with a variety of shapes and sizes. Some of them have lighting effects, while others have the effect of spraying mist in circles, which provides consumers with a wide range of choices. Especially in recent years, the use experience of humidifiers and aromatherapy diffusers is greatly improved by utilizing the structural feature of combining atomization mist components and lighting components.

For example, the invention patent with the application number CN202310080449.2 entitled “AROMATHERAPY DIFFUSER WITH RAINDROP EFFECT” specifically discloses an aromatherapy diffuser with a raindrop effect, which includes a bottom shell, a water tank arranged in the bottom shell and an atomization module arranged on the water tank. A cloud-shaped shell is arranged above the water tank, and the water tank is provided with a water pump module for pumping the liquid inside the water tank into the cloud-shaped shell. The lower end of the cloud-shaped shell is provided with discharge holes, and a light source capable of emitting lights of different colors is also arranged inside the cloud-shaped shell. During the operation, the water contained in the water tank is pumped into the cloud-shaped shell by the water pump module. The water in the cloud-shaped shell is discharged downward from the discharge holes, form raindrops and fall into the water tank, and then be pumped into the cloud-shaped shell by the water pump module again, thus forming a cycle to simulate the scene of raindrops formed by rain falling from clouds. Meanwhile, the light source inside the cloud-shaped shell can emit lights of different colors to simulate the scene of clouds changing colors. In addition, the mist generated by the atomization module floats upward to the lower end or the periphery of the cloud-shaped shell, and forms an effect of simulating a rainbow under the action of the light.

The above-mentioned aromatherapy diffuser utilizes a water pump to pump water to a high position, and then the water drips down, finally achieving an atomization effect similar to a rainbow. However, due to the water pump module in the structure of the above-mentioned prior art product, it not only increases the cost of the product, but also is not conducive to maintenance, and has low ornamental value, which cannot better meet the use needs of consumers.

### SUMMARY

The problem to be solved in the present application in the prior art is as follows:

The aromatherapy diffuser in the prior art generally uses a water pump to pump water to a high position, and then the water drips down, finally achieving an atomization effect similar to a rainbow. However, such product structure has the water pump module, and therefore not only increases the cost of the product but also is not conducive to maintenance, has a low ornamental value, and cannot better meet the use needs of consumers.

The solution to solve the technical problem of the present disclosure is as follows:

Provided is a rotary impeller type aromatherapy humidifier, including a humidifier main body as well as a control circuit board and an atomization assembly which are arranged inside the humidifier main body; the atomization assembly is electrically connected with the control circuit board, wherein
the upper part of the humidifier main body is provided with a mist storage chamber which is communicated with the humidifier main body and used for accumulating mist and condensed water droplets thereof;
the rotary impeller type aromatherapy humidifier further comprises a mist guide cylinder which is used for transporting the mist generated by the atomization assembly to the mist storage chamber; the mist guide cylinder is integrally formed with the mist storage chamber or the upper part of the mist guide cylinder is communicated with the mist storage chamber;
the rotary impeller type aromatherapy humidifier further comprises a rotary impeller which is arranged close to the mist storage chamber and axially rotates; the rotary impeller comprises several water-storable water storage lobes;
the mist storage chamber is provided with a water outlet or the mist storage chamber is connected with a water outlet pipeline, the condensed water droplets of the mist flow into the water storage lobes, and the gravity of the water storage lobes is utilized to drive the continuous axial rotation of the rotary impeller; and
the humidifier main body is provided with a return water air inlet groove which corresponds to the rotary impeller and is used for recovering running water.

Preferably, the bottom of the water storage tank of the humidifier main body is further provided with an atomization energy-gathering cylinder for increasing the height of sprayed mist and water;
the atomization assembly is located at the bottom of the atomization energy-gathering cylinder; and
the outlet on the top of the atomization energy-gathering cylinder is arranged toward the mist guide cylinder.

Preferably, the humidifier main body is provided with a light-emitting diode (LED) lamp or LED lamp plate used for emitting light, or an LED lamp strip is arranged around the outside of the humidifier main body.

Preferably, the humidifier main body includes an outer shell, the water storage tank located on the inner side or upper part of the outer shell and an upper cover located on the upper part of the water storage tank;
the return water air inlet groove is arranged on the upper cover;
the rotary impeller is located on the upper part of the upper cover; and
the lower part of the mist guide cylinder penetrates through the upper cover and then stretches into the inner side of the water storage tank.

Preferably, the upper part of the mist guide cylinder is provided with a water diversion cover plate; the mist storage chamber is formed at the inner side of the water diversion cover plate;

an opening on the upper part of the mist guide cylinder is matched and connected with the water diversion cover plate; and the opening on the upper part of the mist guide cylinder is further provided with a water baffle cover used for preventing the mist and condensed water droplets thereof from being sprayed upward.

Preferably, the upper part of the water diversion cover plate is also matched and provided with a spraying cover; the water diversion cover plate and the spraying cover are integrally arranged or movably connected; and the spraying cover is provided with an air-jet mist outlet.

Preferably, the water outlet is arranged on the water diversion cover plate;

the rotary impeller is movably connected with the outer side of the water diversion cover plate through a rotating shaft.

Preferably, the control circuit board and the atomization assembly are located in an interior space between the bottom of the water storage tank and the outer shell;

the rotary impeller type aromatherapy humidifier further comprises a water-free detection switch arranged inside the humidifier main body; and the water-free detection switch is electrically connected with the control circuit board.

Preferably, the return water air inlet groove is in a strip-shaped structure; and the length of the return water air inlet groove corresponds to the diameter of the rotary impeller to prevent running water from being splashed outward.

Preferably, the mist guide cylinder is in an inverted cone structure whose size gradually increases from bottom to top.

The technical effects generated by solving the technical problems of the present application are as follows:

Compared with the prior art, in the rotary impeller type aromatherapy humidifier provided in the present disclosure, the humidifier main body, the control circuit board 111 and the atomization assembly 12 which are arranged inside the humidifier main body are simultaneously arranged; the atomization assembly 12 is electrically connected with the control circuit board 111, the upper part of the humidifier main body is provided with the mist storage chamber which is communicated with the humidifier main body and used for accumulating mist and condensed water droplets thereof; the rotary impeller type aromatherapy humidifier further includes the mist guide cylinder 16 which is used for transporting the mist generated by the atomization assembly 12 to the mist storage chamber; the mist guide cylinder 16 and the mist storage chamber are integrally formed or the upper part of the mist guide cylinder 16 is communicated with the mist storage chamber; the rotary impeller type aromatherapy humidifier further includes the rotary impeller 18 which is arranged close to the mist storage chamber and axially rotates; the rotary impeller 18 includes several water-storable water storage lobes 181; the mist storage chamber is provided with the water outlet 173 or the mist storage chamber is connected with a water outlet pipeline, the condensed water droplets of the mist flows into the water storage lobes 181, and the gravity of the water storage lobes 181 is utilized to drive the continuous axial rotation of the rotary impeller 18; and the humidifier main body is provided with the return water air inlet groove 151 corresponding to the rotary impeller 18 and used for recovering the running water. In the actual application process, the condensed water droplets of the mist can well fall into the water storage lobes 181, so as to continuously drive of the rotary impeller 18, which not only improves the ornamental value but also has low manufacturing cost, no additional water pump structure and convenient product maintenance.

### BRIEF DESCRIPTION OF THE DRAWINGS

FIG. 1 and FIG. 2 are schematic diagrams of an exploded structure of a rotary impeller type aromatherapy humidifier according to the present disclosure.

FIG. 3 is a schematic diagram of a three-dimensional structure of a rotary impeller type aromatherapy humidifier according to the present disclosure.

FIG. 4 and FIG. 5 are schematic diagrams of a cross-sectional structure of a rotary impeller type aromatherapy humidifier according to the present disclosure in different orientations.

In the figures, outer shell 11; control circuit board 111; atomization assembly 12; water storage tank 13; atomization energy-gathering cylinder 131; LED lamp strip 14; upper cover 15; return water air inlet groove 151; mist guide cylinder 16; water diversion cover plate 17; spraying cover 171; air jet mist outlet 172; water outlet 173; rotary impeller 18; and water storage lobe 181.

### DETAILED DESCRIPTION OF THE EMBODIMENTS

To make the objective, technical solution and advantages of the present disclosure more clear and understandable, the present disclosure will be further described in detail in combination with drawings and embodiments. It should be understood that the specific embodiments described here are merely used for explaining the present disclosure, but not limiting the present disclosure.

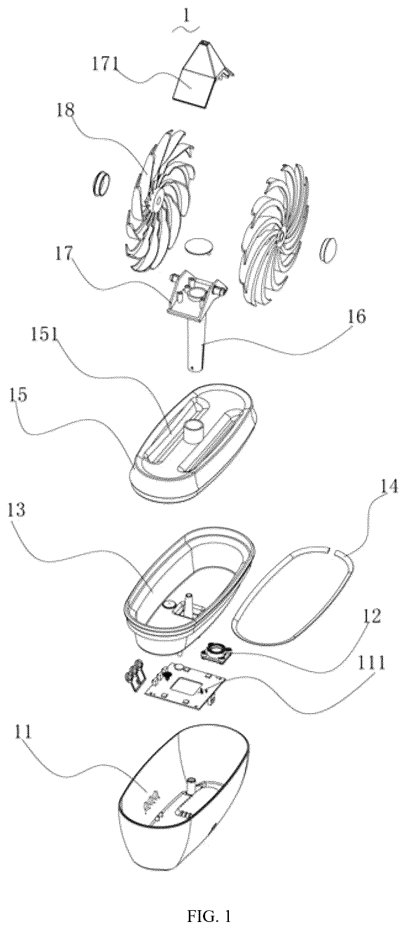

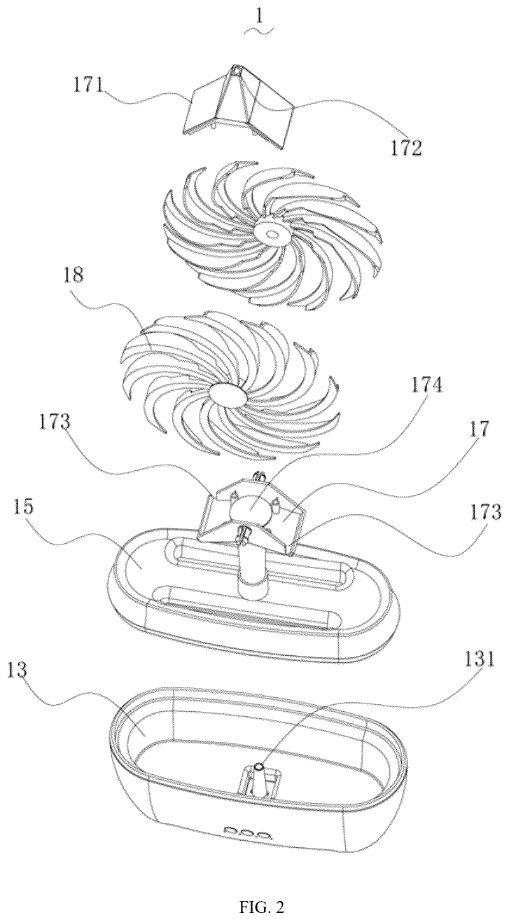

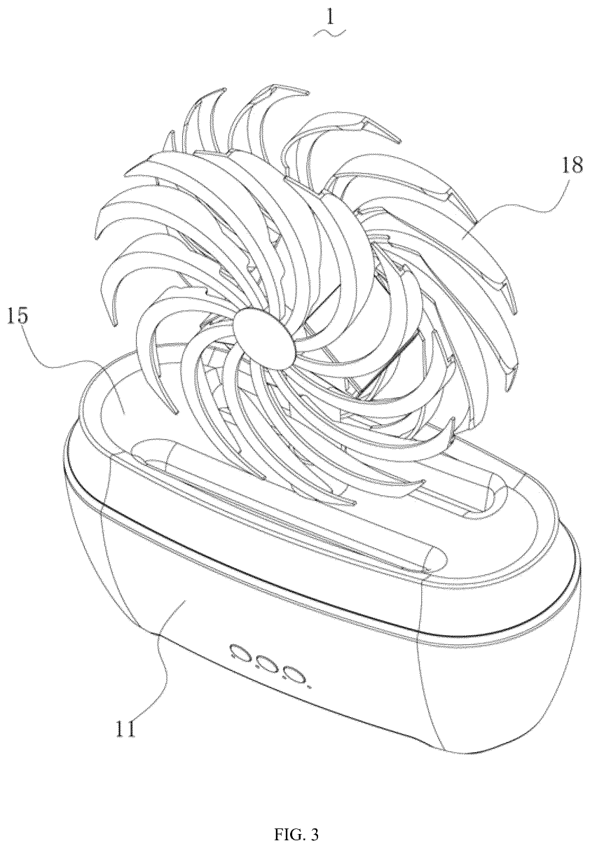

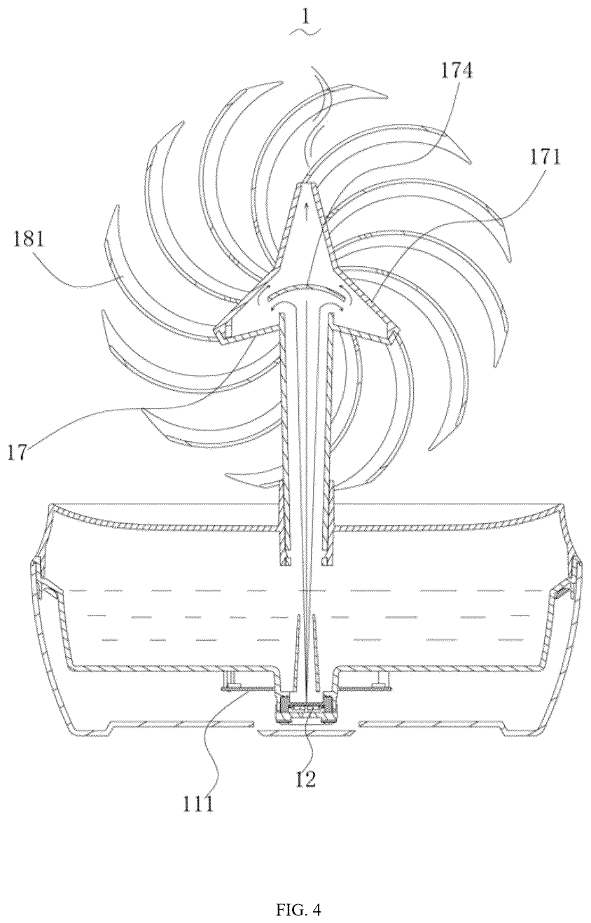

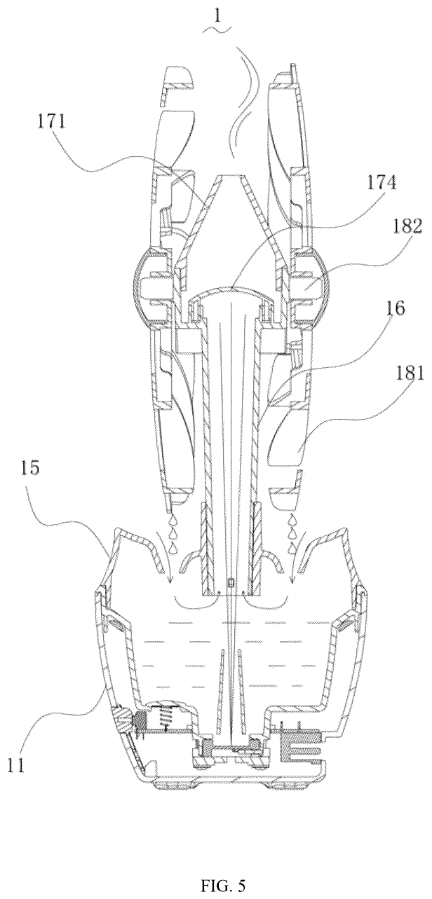

Referring to FIG. 1-FIG. 5, provided is a rotary impeller type aromatherapy humidifier 1, including a humidifier main body, a control circuit board 111 arranged inside the humidifier main body and an atomization assembly 12; the atomization assembly 12 is electrically connected with the control circuit board 111,
the upper part of the humidifier main body is provided with a mist storage chamber which is communicated with the humidifier main body and used for accumulating mist and condensed water droplets thereof;
the rotary impeller type aromatherapy humidifier further includes a mist guide cylinder 16 which is used for transporting the mist generated by the atomization assembly 12 to the mist storage chamber; the mist guide cylinder 16 is integrally formed with the mist storage chamber or the upper part of the mist guide cylinder 16 is communicated with the mist storage chamber;
in actual design, the mist guide cylinder 16 can be in a hollow pipeline structure, or a shell structure matched with the shape of the humidifier main body;
the rotary impeller type aromatherapy humidifier further includes a rotary impeller 18 which is arranged close to the mist storage chamber and axially rotates; the rotary impeller 18 includes several water-storable water storage lobes 181;
the number of the water storage lobes 181 is not specifically limited in the present application, and the water storage lobes 181 mainly function to store water.
the mist storage chamber is provided with a water outlet 173 or the mist storage chamber is connected with the water outlet pipeline, the condensed water droplets of the mist flow into the water storage lobes 181, and the gravity of the water storage lobes 181 is utilized to drive the continuous axial rotation of the rotary impeller 18; and
the humidifier main body is provided with a return water air inlet groove 151 which corresponds to the rotary impeller 18 and is used for recovering running water.

In the present application, the humidifier main body as well as the control circuit board 111 and the atomization assembly 12 which are arranged inside the humidifier main body are simultaneously arranged; the atomization assembly 12 is electrically connected with the control circuit board 111, the upper part of the humidifier main body is provided with the mist storage chamber which is communicated with the humidifier main body and used for accumulating mist and condensed water droplets thereof; the rotary impeller type aromatherapy humidifier further includes the mist guide cylinder 16 which is used for transporting the mist generated by the atomization assembly 12 to the mist storage chamber; the mist guide cylinder 16 and the mist storage chamber are integrally formed or the upper part of the mist guide cylinder 16 is communicated with the mist storage chamber; the rotary impeller type aromatherapy humidifier further includes the rotary impeller 18 which is arranged close to the mist storage chamber and axially rotates; the rotary impeller 18 includes several water-storable water storage lobes 181; the mist storage chamber is provided with the water outlet 173 or the mist storage chamber is connected with the water outlet pipeline, the condensed water droplets of the mist flows into the water storage lobes 181, and the gravity of the water storage lobes 181 is utilized to drive the continuous axial rotation of the rotary impeller 18; and the humidifier main body is provided with the return water air inlet groove 151 corresponding to the rotary impeller 18 and used for recovering the running water. In the actual application process, the condensed water droplets of the mist can well fall into the water storage lobes 181, so as to continuously drive of the rotary impeller 18, which not only improves the ornamental value but also has low manufacturing cost, no additional water pump structure and convenient product maintenance.

In some other embodiments, the bottom of the water storage tank 13 of the humidifier main body is further provided with an atomization energy-gathering cylinder 131 used for increasing the height of sprayed mist and water;
the atomization assembly 12 is located at the bottom of the atomization energy-gathering cylinder 131; and
the outlet on the top of the atomization energy-gathering cylinder 131 is arranged toward the mist guide cylinder 16.

The humidifier main body is internally provided with an LED lamp or LED lamp plate used for emitting light, or an LED lamp strip 14 is arranged around the outside of the humidifier main body.

The humidifier main body includes an outer shell 11, the water storage tank 13 located on the inner side or upper part of the outer shell 11 and an upper cover 15 located on the upper part of the water storage tank 13;
the return water air inlet groove 151 is arranged on the upper cover 15;
the rotary impeller 18 is located on the upper part of the upper cover 15; and
the lower part of the mist guide cylinder 16 penetrates through the upper cover 15 and then stretches into the inner side of the water storage tank 13.

The upper part of the mist guide cylinder 16 is provided with a water diversion cover plate 17; the mist storage chamber is formed at the inner side of the water diversion cover plate 17;

an opening on the upper part of the mist guide cylinder 16 is matched and connected with the water diversion cover plate 17; and the opening on the upper part of the mist guide cylinder 16 is further provided with a water baffle cover 174 used for preventing the mist and condensed water droplets thereof from being sprayed upward.

The upper part of the water diversion cover plate 17 is also matched and provided with a spraying cover 171; the water diversion cover plate 17 and the spraying cover 171 are integrally arranged or movably connected; and

the spraying cover 171 is provided with an air-jet mist outlet 172.

The water outlet 173 is arranged on the water diversion cover plate 17;

In actual design, the water outlet 173 is arranged at left and right sides of the water diversion cover plate 17;

the rotary impeller 18 is movably connected with the outer side of the water diversion cover plate 17 through a rotating shaft 182.

The control circuit board 111 and the atomization assembly 12 are located in an interior space between the bottom of the water storage tank 13 and the outer shell 11;

the rotary impeller type aromatherapy humidifier further includes a water-free detection switch arranged inside the humidifier main body; and the water-free detection switch is electrically connected with the control circuit board 111.

The return water air inlet groove 151 is in a strip-shaped structure; and the length of the return water air inlet groove 151 corresponds to the diameter of the rotary impeller 18 to prevent running water from being splashed outward.

The mist guide cylinder 16 is in an inverted cone structure whose size gradually increases from bottom to top.

The operation principle is as follows:

This design uses the water sprayed by the atomizing sheet during the operation to be transported to the water diversion cover plate 17, and then flows from the water outlet 173 on the water diversion cover plate 17 to the water storage lobes 181 of the rotary impeller 18. The water on the lobes increases the gravity so that the impeller obtains the power to rotate. In addition, the mist atomized by the atomizing sheet drifts out from the air-jet mist outlet 172 to form mist, and the lighting effect is added to achieve a visual ornamental effect.

The technical effects generated by solving the technical problems of the present application are as follows:

Compared with the prior art, in the rotary impeller type aromatherapy humidifier 1, the humidifier main body as well as the control circuit board 111 and the atomization assembly 12 which are arranged inside the humidifier main body are simultaneously arranged; the atomization assembly 12 is electrically connected with the control circuit board 111, the upper part of the humidifier main body is provided with the mist storage chamber which is communicated with the humidifier main body and used for accumulating mist and condensed water droplets thereof; the rotary impeller type aromatherapy humidifier further includes the mist guide cylinder 16 which is used for transporting the mist generated by the atomization assembly 12 to the mist storage chamber; the mist guide cylinder 16 and the mist storage chamber are integrally formed or the upper part of the mist guide cylinder 16 is communicated with the mist storage chamber; the rotary impeller type aromatherapy humidifier further includes the rotary impeller 18 which is arranged close to the mist storage chamber and axially rotates; the rotary impeller 18 includes several water-storable water storage lobes 181; the mist storage chamber is provided with the water outlet 173 or the mist storage chamber is connected with the water outlet pipeline, the condensed water droplets of the mist flows into the water storage lobes 181, and the gravity of the water storage lobes 181 is utilized to drive the continuous axial rotation of the rotary impeller 18; and the humidifier main body is provided with the return water air inlet groove 151 corresponding to the rotary impeller 18 and used for recovering the running water. In the actual application process, the condensed water droplets of the mist can well fall into the water storage lobes 181, so as to continuously drive of the rotary impeller 18, which not only improves the ornamental value but also has low manufacturing cost, no additional water pump structure and convenient product maintenance.

The aforementioned embodiments of the present disclosure are not intended to limit the protective scope of the present disclosure. Any amendments, equivalent replacements, improvements and the like made within the spirit and principle of the present disclosure all should be included within the protective scope of the appended claims of the present disclosure.

What is claimed is:

1. A rotary impeller type aromatherapy humidifier, comprising a humidifier main body as well as a control circuit board and an atomization assembly which are arranged inside the humidifier main body; the atomization assembly being electrically connected with the control circuit board, wherein
the upper part of the humidifier main body is provided with a mist storage chamber which is communicated with the humidifier main body and used for accumulating mist and condensed water droplets thereof;
the rotary impeller type aromatherapy humidifier further comprises a mist guide cylinder which is used for transporting the mist generated by the atomization assembly to the mist storage chamber; the mist guide cylinder is integrally formed with the mist storage chamber or the upper part of the mist guide cylinder is communicated with the mist storage chamber;
the rotary impeller type aromatherapy humidifier further comprises a rotary impeller which is arranged close to the mist storage chamber and axially rotates; the rotary impeller comprises several water-storable water storage lobes;
the mist storage chamber is provided with a water outlet or the mist storage chamber is connected with a water outlet pipeline, the condensed water droplets of the mist flow into the water storage lobes, and the gravity of the water storage lobes is utilized to drive the continuous axial rotation of the rotary impeller; and
the humidifier main body is provided with a return water air inlet groove which corresponds to the rotary impeller and is used for recovering running water.

2. The rotary impeller type aromatherapy humidifier according to claim 1, wherein the bottom of the water storage tank of the humidifier main body is further provided with an atomization energy-gathering cylinder for increasing the height of sprayed mist and water;
the atomization assembly is located at the bottom of the atomization energy-gathering cylinder; and
the outlet on the top of the atomization energy-gathering cylinder is arranged toward the mist guide cylinder.

3. The rotary impeller type aromatherapy humidifier according to claim 1, wherein the humidifier main body is internally provided with a light-emitting diode (LED) lamp or LED lamp plate used for emitting light or an LED lamp strip is arranged around the outside of the humidifier main body.

4. The rotary impeller type aromatherapy humidifier according to claim 1, wherein the humidifier main body comprises an outer shell, the water storage tank located on the inner side or upper part of the outer shell and an upper cover located on the upper part of the water storage tank;
the return water air inlet groove is arranged on the upper cover;
the rotary impeller is located on the upper part of the upper cover; and
the lower part of the mist guide cylinder penetrates through the upper cover and then stretches into the inner side of the water storage tank.

5. The rotary impeller type aromatherapy humidifier according to claim 1, wherein the upper part of the mist guide cylinder is provided with a water diversion cover plate; the mist storage chamber is formed at the inner side of the water diversion cover plate;
an opening on the upper part of the mist guide cylinder is matched and connected with the water diversion cover plate; and the opening on the upper part of the mist guide cylinder is further provided with a water baffle cover used for preventing the mist and condensed water droplets thereof from being sprayed upward.

6. The rotary impeller type aromatherapy humidifier according to claim 5, wherein the upper part of the water diversion cover plate is also matched and provided with a spraying cover; the water diversion cover plate and the spraying cover are integrally arranged or movably connected; and
the spraying cover is provided with an air-jet mist outlet.

7. The rotary impeller type aromatherapy humidifier according to claim 5, wherein the water outlet is arranged on the water diversion cover plate; and
the rotary impeller is movably connected with the outer side of the water diversion cover plate through a rotating shaft.

8. The rotary impeller type aromatherapy humidifier according to claim 4, wherein the control circuit board and the atomization assembly are located in an interior space between the bottom of the water storage tank and the outer shell;
the rotary impeller type aromatherapy humidifier further comprises a water-free detection switch arranged inside the humidifier main body; and the water-free detection switch is electrically connected with the control circuit board.

9. The rotary impeller type aromatherapy humidifier according to claim 4, wherein the return water air inlet groove is in a strip-shaped structure; and the length of the return water air inlet groove corresponds to the diameter of the rotary impeller to prevent running water from being splashed outward.

10. The rotary impeller type aromatherapy humidifier according to claim 1, wherein the mist guide cylinder is in an inverted cone structure whose size gradually increases from bottom to top.
